(19) United States
(12) Patent Application Publication
(10) Pub. No.: US 2024/0238479 A1
(43) Pub. Date: Jul. 18, 2024

(54) COLLAGEN AND SODIUM HYALURONATE CROSS-LINKED COMPOSITE FILLER FOR INJECTION AND PREPARATION METHOD THEREOF

(71) Applicant: Hangzhou Singclean Medical Products Co., Ltd, Hangzhou (CN)

(72) Inventors: Qingzhi Deng, Hangzhou (CN); Zhihong Zhang, Hangzhou (CN); Yufei Xiong, Hangzhou (CN); Wei Huang, Hangzhou (CN); Quan Zhang, Hangzhou (CN); Bair Zu, Hangzhou (CN)

(21) Appl. No.: 18/135,733

(22) Filed: Apr. 17, 2023

(30) Foreign Application Priority Data
Jan. 18, 2023 (CN) 202310055672.1

(51) Int. Cl. A61L 27/26

(52) U.S. Cl. CPC A61L 27/26; A61L 2400/06; A61L 2430/34

(57) ABSTRACT

The present disclosure provides a collagen and sodium hyaluronate cross-linked composite filler for injection and a preparation method thereof. The collagen and sodium hyaluronate cross-linked composite filler is obtained by a cross-linking reaction of collagen and sodium hyaluronate with a 1-(3-dimethylaminopropyl)-3-ethylcarbodiimide hydrochloride/N-hydroxysuccinimide (EDC/NHS) cross-linking system. As the EDC/NHS is used as a cross-linking agent in the present disclosure, the resulting product has no biological toxicity, so that the injury caused by a final product to an injection site and the human body is reduced. Meanwhile, due to intramolecular and intermolecular cross-linking, the degradation rate of the collagen and the sodium hyaluronate is reduced, and the retention time of the two in the body is prolonged. Therefore, the collagen and sodium hyaluronate cross-linked composite filler for injection in the present disclosure can be used in plastic surgery in the medical industry.

### CROSS-REFERENCE TO RELATED APPLICATIONS

The present application claims the benefit of Chinese Patent Application No. 202310055672.1 filed on Jan. 18, 2023, the contents of which are incorporated by reference herein in their entirety.

### TECHNICAL FIELD

The present disclosure relates to the field of medical bioengineering, and in particular to a collagen and sodium hyaluronate cross-linked composite filler for injection and a preparation method thereof.

### BACKGROUND

With the development of society and the improvement of living standards, people have a further pursuit of physical beauty on the premise of keeping healthy. Especially with the increase of age, the skin has a series of problems, such as dehydration, loss of elasticity, and wrinkles. As conventional skin care products can no longer satisfy people's pursuit of beauty, cosmetic surgery is emerged and developed rapidly. Among various cosmetic surgery technologies, cosmetic injection is favored by the majority of those who care their beauty due to quick and obvious effects, especially small injury to the body and other advantages. Collagen and hyaluronic acid materials are undoubtedly two “favorites” in the technical field of cosmetic surgery.

Hyaluronic acid, also known as HA, is a high-grade polysaccharide consisting of D-glucuronic acid and N-acetylglucosamine. The hyaluronic acid materials are widely found in the human body and have high biocompatibility. Sodium hyaluronate has a strong moisturizing ability and can hold up to water molecules 1,000 times its weight, thus having a reputation of the “strongest moisturizer”. The sodium hyaluronate has the effects of moisturizing, relieving wrinkles and maintaining skin luster when applied in the fields of skin care products and medical beauty. At present, the injection of a sodium hyaluronate solution has been widely used for hydration and moisturizing on the market. The skin becomes significantly better 7 days after the injection, and becomes smooth and bright, with pores becoming smaller. The market satisfaction degree and the repurchase rate are very high. The effects can generally be maintained for about 1-3 months.

The collagen is a biopolymer material widely found in the bodies of mammals, and is an extremely important main component in connective tissues and also a main component in extracellular matrices. With the increase of age, the collagen on the face of the human body is also gradually lost, and the skin will have a series of unattractive phenomena, such as appearance of nasolabial folds, loss of elasticity, and wrinkles. Thus, direct injection of the collagen is the fastest method to achieve effects.

Attempts have been made to compound the collagen and the sodium hyaluronate to improve the effects of the two and to achieve a synergistic effect. However, the collagen and the sodium hyaluronate are two incompatible systems, which will have precipitation after compounding. If the two can be connected by a material to form large molecules, which are then uniformly dispersed in a solution, two different effects can be achieved at the same time.

### SUMMARY

An objective of the present disclosure is to develop a cross-linking method for stably and firmly connecting collagen and sodium hyaluronate to obtain a collagen and sodium hyaluronate cross-linked composite filler for injection.

In order to realize the above objective, the following technical solutions are adopted in the present disclosure according to a first aspect of the present disclosure.

A collagen and sodium hyaluronate cross-linked composite filler for injection is prepared by a cross-linking reaction of collagen and sodium hyaluronate with a carbodiimide type cross-linking agent. The carbodiimide type cross-linking agent is 1-(3-dimethylaminopropyl)-3-ethylcarbodiimide hydrochloride/N-hydroxysuccinimide (EDC/NHS).

The NHS is used as an activator, the EDC is used as a cross-linking agent, and the ratio of the two is 1:1 to 10:1 (molar ratio).

Preferably, the collagen is animal type I collagen or type III collagen.

Preferably, the sodium hyaluronate is obtained by bacterial fermentation, and has a molecular weight ranging from 100,000 to 3 million Da.

In another aspect, the present disclosure also provides a preparation method of the above collagen and sodium hyaluronate cross-linked composite filler for injection. The method includes the following steps:
(1) dissolving the collagen in an acetic acid aqueous solution to obtain a solution A with a collagen concentration of 5-10 mg/mL; and dissolving the sodium hyaluronate in water, and conducting stirring for 5-17 h to obtain a solution B with a sodium hyaluronate concentration of 5-10 mg/mL;
(2) mixing the solution A with the solution B at a ratio of 1:1 to 3:1 (volume ratio), conducting stirring for 1-5 h for uniform mixing, and adjusting the pH to 4.5 to 5.5;
(3) adding the cross-linking agent for a cross-linking reaction, namely, adding 0.06 g/L to 60 g/L activator (NHS) first, uniformly stirring, and then adding 0.1 g/L to 10 g/L cross-linking agent (EDC) for the cross-linking reaction at a temperature of 20-30° C. for 8-30 h;
(4) centrifuging a resulting product and cleaning with a cleaning agent to remove the cross-linking agent, the cleaning agent being a phosphate buffer solution with a pH of 7 to 9;
(5) freeze-drying the cleaned product to obtain a white solid; and
(6) dissolving the obtained white solid in a phosphate buffer solution (pH=6.8 to 7.2) at a concentration of 3-5%, adding 1% to 5% suspension agent (sodium carboxymethyl cellulose) for dissolution, and then conducting emulsification and homogenization to obtain the collagen and sodium hyaluronate cross-linked composite filler.

The collagen and sodium hyaluronate cross-linked composite filler for injection prepared by the present disclosure has the following beneficial effects.

As the EDC/NHS is used as a cross-linking agent in the present disclosure, the resulting product has no biological toxicity, so that the injury caused by a final product to an injection site and the human body is reduced. Meanwhile, due to intramolecular and intermolecular cross-linking, the degradation rate of the collagen and the sodium hyaluronate is reduced, and the retention time of the two in the body is prolonged. Therefore, the collagen and sodium hyaluronate cross-linked composite filler for injection in the present disclosure can be used in plastic surgery in the medical industry.

### DETAILED DESCRIPTION

In order to make the solutions of the present disclosure better understood by a person in the art, the present disclosure is further described in detail below with specific embodiments.

### Example 1

(1) An animal tissue, bovine achilles tendon, was subjected to slicing, cleaning, acid hydrolysis, enzyme hydrolysis, salting out, purification and other steps to obtain high-purity collagen, namely type I collagen.

(2) The type I collagen, as a water-insoluble fibrous protein was dissolved by adding 0.1% (v/v) acetic acid aqueous solution in an intermediate reaction process to obtain a collagen solution A with a pH of about 4 (pH of 4 to 5) and a collagen concentration of 6 mg/ml.

(3) Sodium hyaluronate with a molecular weight of 2.3 million Da was used and dissolved in water by stirring at room temperature for 12 h to obtain a sodium hyaluronate solution B with a concentration of 6 mg/ml.

(4) The solution A was mixed with the solution B at a ratio of 1:1 (volume ratio), the pH was adjusted to 5.0 with 0.2 mol/L disodium hydrogen phosphate solution, and stirring was conducted at room temperature for 1 h.

(5) An activator (NHS) was added to make the concentration to 0.2 g/L, stirring was conducted for 5 min, then a cross-linking agent (EDC) was added to make the final concentration of EDC to 5 g/L, and a reaction was carried out at 25° C. for 10 h. After centrifugation was conducted, cross-linked filamentous fibers were collected.

(6) The cross-linked products obtained after the centrifugation were cleaned 5 times with a 5 times phosphate buffer solution. The pH of the phosphate buffer solution used for cleaning was 8. With the phosphate buffer solution being used as a cleaning agent, the cross-linked fiber products may be retained at current situations without degradation. (a formula of the phosphate buffer solution: 1 L of water, 0.4 g of sodium dihydrogen phosphate, and 1.1 g of disodium hydrogen phosphate).

(7) After centrifugation was conducted, a supernatant was poured, and a solid in the lower layer was collected and freeze-dried at low temperature to obtain a white solid.

(8) The obtained white solid was dissolved in a phosphate buffer solution (pH=7.0) at a concentration of 3%, 2% sodium carboxymethyl cellulose was added and stirred uniformly, and then emulsification and homogenization were conducted at 2,000 r/min for 10 min.

(9) A milky white solution having viscoelasticity was obtained after the homogenization, and then charged into a syringe to obtain a collagen and sodium hyaluronate cross-linked composite filler for injection.

### Example 2

(1) An animal tissue, pig skin, was subjected to slicing, acid hydrolysis, enzyme hydrolysis, salting out, purification and other steps to obtain high-purity type I collagen.

(2) The type I collagen, as a water-insoluble fibrous protein, was dissolved by adding 0.1% (v/v) acetic acid aqueous solution in an intermediate reaction process to obtain a collagen solution A with a pH of about 4 (pH of 4 to 5) and a collagen concentration of 7 mg/ml.

(3) Sodium hyaluronate with a molecular weight of 1.8 million Da was used and dissolved in water by stirring at room temperature for 8 h to obtain a sodium hyaluronate solution B with a concentration of 8 mg/ml.

(4) The solution A was mixed with the solution B at a ratio of 2:1 (volume ratio), the pH was adjusted to 5.0 with 0.2 mol/L disodium hydrogen phosphate solution, and stirring was conducted at room temperature for 1.5 h.

(5) An activator (NHS) was added to make the concentration to 2 g/L, stirring was conducted for 5 min, then a cross-linking agent (EDC) was added to make the final concentration of EDC to 8 g/L, and a reaction was carried out at 25° C. for 12 h. After centrifugation was conducted, cross-linked filamentous fibers were collected.

(6) The cross-linked products obtained after the centrifugation were cleaned 4 times with a 3 times phosphate buffer solution. The pH of the phosphate buffer solution used for cleaning was 8.5. With the phosphate buffer solution being used as a cleaning agent, the cross-linked fiber products may be retained at current situations without degradation. (a formula of the phosphate buffer solution: 1 L of water, 0.4 g of sodium dihydrogen phosphate, and 1.2 g of disodium hydrogen phosphate).

(7) After centrifugation was conducted, a supernatant was poured, and a solid in the lower layer was collected and freeze-dried at low temperature to obtain a white solid.

(8) The obtained white solid was dissolved in a phosphate buffer solution (pH=6.9) at a concentration of 4%, 2% sodium carboxymethyl cellulose was added and stirred uniformly, and then emulsification and homogenization were conducted at 2,000 r/min for 10 min.

(9) A milky white solution having viscoelasticity was obtained after the homogenization, and then charged into a syringe to obtain a collagen and sodium hyaluronate cross-linked composite filler for injection.

### Example 3

(1) An animal tissue, bovine achilles tendon, was subjected to slicing, acid hydrolysis, enzyme hydrolysis, salting out, purification and other steps to obtain high-purity type I collagen.

(2) The type I collagen, as a water-insoluble fibrous protein, was dissolved by adding 0.1% (v/v) acetic acid aqueous solution in an intermediate reaction process to obtain a collagen solution A with a pH of about 4 (pH of 4 to 5) and a collagen concentration of 7 mg/ml.

(3) Sodium hyaluronate with a molecular weight of 800,000 Da was used and dissolved in water by stirring at room temperature for 5 h to obtain a sodium hyaluronate solution B with a concentration of 9 mg/ml.

(4) The solution A was mixed with the solution B at a ratio of 3:1 (volume ratio), the pH was adjusted to 5.0 with 0.2 mol/L disodium hydrogen phosphate solution, and stirring was conducted at room temperature for 2 h.

(5) An activator (NHS) was added to make the concentration to 3 g/L, stirring was conducted for 5 min, then a cross-linking agent (EDC) was added to make the final concentration of EDC to 9 g/L, and a reaction was carried out at 25° C. for 12 h. After centrifugation was conducted, cross-linked filamentous fibers were collected.

(6) The cross-linked products obtained after the centrifugation were cleaned 5 times with a 3 times phosphate buffer solution. The pH of the phosphate buffer solution used for cleaning was 8.5. With the phosphate buffer solution being used as a cleaning agent, the cross-linked fiber products may be retained at current situations without degradation. (a formula of the phosphate buffer solution: 1 L of water, 0.4 g of sodium dihydrogen phosphate, and 1.2 g of disodium hydrogen phosphate).

(7) After centrifugation was conducted, a supernatant was poured, and a solid in the lower layer was collected and freeze-dried at low temperature to obtain a white solid.

(8) The obtained white solid was dissolved in a phosphate buffer solution (pH=6.9) at a concentration of 5%, 1% sodium carboxymethyl cellulose was added and stirred uniformly, and then emulsification and homogenization were conducted at 2,000 r/min for 10 min.

(9) A milky white solution having viscoelasticity was obtained after the homogenization, and then charged into a syringe to obtain a collagen and sodium hyaluronate cross-linked composite filler for injection.

The yield, cross-linking degree, stability and viscoelasticity of the cross-linked products obtained in Examples 1 to 3 were determined. Results are as shown in Table 1.

TABLE 1
Yield, cross-linking degree, stability and viscoelasticity
of the cross-linked products obtained in Examples 1 to 3

| | | Cross-linking | | Viscoelasticity | | |
|---|---|---|---|---|---|---|
| | Yield | degree | | Elastic | Elastic | |
| | (%) | (%) | Stability | modulus | modulus | |
| Example 1 | 62 | 58 | No obvious change after | 308 pa | 101 | pa |
| | | | standing for 3 months | | | |
| Example 2 | 68 | 52 | No obvious change after | 212 pa | 88 | pa |
| | | | standing for 3 months | | | |
| Example 3 | 66 | 49 | No obvious change after | 157 pa | 64 | pa |
| | | | standing for 3 months | | | |

From Table 1, it can be seen that by using the preparation method provided by the present disclosure, effective chemical cross-linking of the collagen and the sodium hyaluronate can be realized, and the obtained product has good stability and suitable viscoelasticity and meets injection conditions.

What is claimed is:

1. A preparation method of a collagen and sodium hyaluronate cross-linked composite filler for injection, comprising the following steps:
(1) dissolving the collagen in an acetic acid aqueous solution to obtain a solution A with a collagen concentration of 5-10 mg/mL; and dissolving the sodium hyaluronate in water to obtain a solution B with a sodium hyaluronate concentration of 5-10 mg/mL;
(2) mixing the solution A with the solution B at a ratio of 1:1 to 3:1 (volume ratio), conducting stirring for uniform mixing, and adjusting the pH to 4.5 to 5.5;
(3) adding a cross-linking agent for a cross-linking reaction, namely, adding 0.06 g/L to 60 g/L activator (NHS) first, conducting uniform stirring, and then adding 0.1 g/L to 10 g/L cross-linking agent (EDC) for a cross-linking reaction at a temperature of 20-30° C. for 8-30 h, the ratio of the NHS to the EDC being 1:1 to 10:1 (molar ratio);
(4) subjecting a resulting product to centrifugation and cleaning with a cleaning agent to remove the cross-linking agent, the cleaning agent being a phosphate buffer solution with a pH of 7 to 9;
(5) drying the cleaned product to obtain a white solid; and
(6) dissolving the obtained white solid in a phosphate buffer solution (pH=6.8 to 7.2) at a concentration of 3-5%, adding 1% to 5% suspension agent (sodium carboxymethyl cellulose) for dissolution, and then conducting emulsification and homogenization to obtain the collagen and sodium hyaluronate cross-linked composite filler.

2. The preparation method of claim 1, wherein the collagen is animal type I collagen or type III collagen.

3. The preparation method of claim 1, wherein the sodium hyaluronate is obtained by bacterial fermentation, and has a molecular weight ranging from 100,000 to 3 million Da.
